(19) United States
(12) Patent Application Publication
(10) Pub. No.: US 2018/0353159 A1
(43) Pub. Date: Dec. 13, 2018

(54) CALIBRATION OF TWO SYNCHRONIZED MOTION PICTURES FROM MAGNETOCARDIOGRAPHY AND ECHOCARDIOGRAPHY

(71) Applicant: Xuan Zhong Ni, santa rosa, CA (US)

(72) Inventor: Xuan Zhong Ni, santa rosa, CA (US)

(21) Appl. No.: 15/619,800

(22) Filed: Jun. 12, 2017

(51) Int. Cl. A61B 8/08; H04N 13/02; G06T 7/20

(52) U.S. Cl. CPC A61B 8/5261; G06T 2207/30048; G06T 7/20; H04N 13/0246

(57) ABSTRACT

A method of calibration for combining two synchronized motion pictures from Magnetocardiography (MCG) and Echocardiography (ECOCG) is accomplished here.

To combine the two motion pictures of MCG and ECOCG is to locate the current dipole sources from a MCG onto a simultaneously beating heart images from an ECOCG. For this purpose, it needs to calibrate the space locations and orientations for the two motion pictures.

In embodiments, the calibration of the spaces and orientation is accomplished by choosing two specific events during a cardiac cycle when the space locations and the orientations of the events for both the MCG and the ECOCG are relatively easy to be determined. Then the two pairs of corresponding space points can be lined up by a coordinate transformation.

This is a non-provisional patent application based on the provisional patent application Ser. No. 62/379,622, filed on Aug. 25, 2016.

### REFERENCES CITED

### U.S. Patent Documents

62/247,778 January 2016 X. Ni

U.S. Pat. No. 8,406,848 B2 March 2013 Wu et al.

U.S. Pat. No. 8,553,956 B2 August 2013 Wu et al.

### BACKGROUND

A magnetocardiography (MCG) motion picture is derived by measuring the magnetic fields at 6×6 locations on a same plane above the heart by very sensitive sensors of the MCG.

The measured magnetic field data are used to reconstruct the current dipole sources of interest. The space positions and orientations of the current dipoles can be determined in certain precision related to the fixed sensors plane of the MCG above the heart.

An echocardiography (ECOCG) motion picture is derived by an ECOCG using ultrasonic scanning. The beating heart images can be seen very clearly frame by frame during a cardiac circle. To locate the current dipole sources from the MCG onto the heart images of the ECOCG will be useful for the doctors.

The difficulty is that the space locations and orientations of the heart images from the ECOCG are uncertain due to the operation.

To locate the current dipole sources from a MCG onto a simultaneously beating heart images from an ECOCG, it needs to calibrate the space locations and orientations for the two motion pictures.

### SUMMARY OF THE INVENTION

A method of calibration of two motion pictures from MCG and ECOCG is accomplished here.

To combine the two motion pictures of MCG and ECOCG it needs to locate the current dipole sources from a MCG onto a simultaneously beating heart images from an ECOCG. For this purpose, it needs to calibrate the space locations and orientations of the two motion pictures.

In embodiments, the calibration of the spaces and orientation is accomplished by choosing two specific events during a cardiac cycle when the space locations and the orientations of the events for both the MCG and the ECOCG are relatively easy to be determined. Then the two pairs of corresponding space points can be lined up by a coordinate transformation.

### BRIEF DESCRIPTION OF THE DRAWINGS

FIG. 1 shows a cardiac cycle of conventional PQRST waves from Electrocardiography.

FIG. 2 shows the t₁ frame of a cardiac cycle from a motion picture of ECOCG.

FIG. 3 shows the t₂ frame of a cardiac cycle from a motion picture of ECOCG.

FIG. 4 shows the t₁ frame of a cardiac cycle from a motion picture of MCG.

FIG. 5 shows the t₂ frame of a cardiac cycle from a motion picture of MCG.

### DETAILED DESCRIPTION OF THE PREFERRED EMBODIMENTS

A method of calibration of two synchronized motion pictures of MCG and ECOCG is accomplished here.

To combine the two motion pictures of MCG and ECOCG it needs to locate the current dipole sources from a MCG onto a simultaneously beating heart images from an ECOCG. For this purpose, it needs to calibrate the space locations and orientations for the two motion pictures.

In embodiments, the calibration of the spaces and orientation is accomplished by choosing two specific events during a cardiac cycle when the space locations and the orientations of the events for both the MCG and the ECOCG are relatively easy to be determined. Then the two pairs of corresponding space points can be lined up by a coordinate transformation.

The MCG motion picture is derived by measuring the magnetic fields at 6×6 locations on a same plane above the heart by very sensitive sensors of the MCG.

The magnetic field data are used to reconstruct the current dipole sources of interest. The space positions and orientations of the current dipoles vectors can be determined in certain precision related to the fixed sensors plane of the MCG above the heart.

An ECOCG motion picture is derived by an ECOCG by ultrasonic scanning. The beating heart images can be seen frame by frame clearly during a cardiac circle. But the space position and orientation of the heart images from the echocardiography are uncertain due to the operation. In order to locate the current dipole vectors it needs to calibrate the two motion pictures.

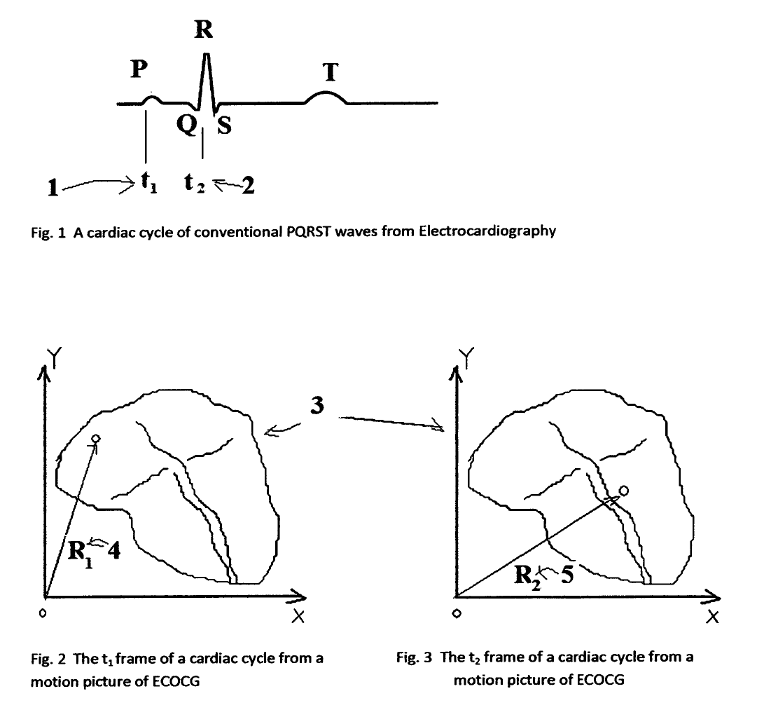

From a normal Electrocardiography picture as in FIG. 1, the P-wave 1 and R-wave 2 have larger magnitudes during a cardiac cycle. The space information of the bioelectric activities have been extensively studied and well understood for these two waves. Hence we choose the two events as the on-setting of P-wave and R-wave with the corresponding timing is t₁ and t₂.

Now we take a look of the t₁ frame and t₂ frame from the ECOCG motion pictures as shown in FIG. 2 and FIG. 3 of the beating heart images 3. It can be located of space positions, R₁ 4 as near the Sinoatrial Node, and R₂ 5 as in the center of left Ventricle closed to the segment.

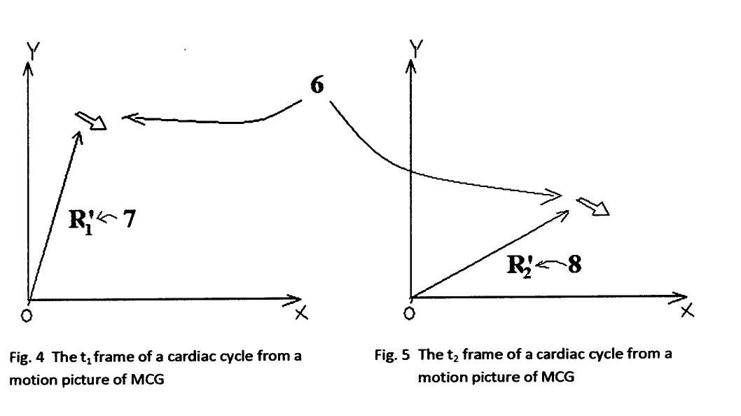

Correspondingly we take a look of the t₁ frame and t₂ frame from the MCG motion pictures as shown in FIG. 4 and FIG. 5 of the current dipole sources 6 from the magnetic measurement. It can be located of space positions, R₁′7 as near the Sinoatrial Node, and R₂′8 as in the center of left Ventricle closed to the segment.

Here it needs to calibration the coordinate system of ECOCG with that of MCG by letting the pair points (R₁, R₁′) be the same physical point, same as the pair (R₂, R₂′).

The coordinate transformation from MCG to ECOCG is as following;

(R−R₂)ₓ=k(cos α(R′−R₂′)ₓ+sin α(R′−R₂′)_{y})

(R−R₂)_{y}=k(−sin α(R′−R₂′)ₓ+cos α(R′−R₂′)_{y})

Here, α=tg⁻¹((R₁−R₂)_{y}/(R₁−R₂)ₓ)−tg⁻¹((R₁′−R₂′)_{y}/(R₁′−R₂′)ₓ) is the angle of rotation, and k=(|R₁−R₂|)/(|R₁′−R₂′|) is the scaling factor.

What is claimed is:

1. A computer program product to execute a method of calibration of two synchronized motion pictures from the Echocardiography and Magnetocardiography.

2. A method of choosing two specific events in a cardiac cycle for calibration of claim 1.

3. A method of choosing P-wave and R-wave as the two specific events of claim 2.

4. A coordinate transformation of Calibration of claim 1.
